(19) United States
(12) Patent Application Publication
(10) Pub. No.: US 2023/0160020 A1
(43) Pub. Date: May 25, 2023

(54) Methods and Systems for Analyzing and Utilizing Cancer Testis Antigen Burden

(71) Applicant: OmniSeq, Inc., Buffalo, NY (US)

(72) Inventors: Sarabjot Pabla, Buffalo, NY (US); Robert J. Seager, JR., Buffalo, NY (US); Erik Van Roey, Buffalo, NY (US); Shuang Gao, Buffalo, NY (US); Jeffrey M. Conroy, Williamsville, NY (US)

(21) Appl. No.: 18/055,292

(22) Filed: Nov. 14, 2022

Related U.S. Application Data
(60) Provisional application No. 63/263,913, filed on Nov. 11, 2021.

(51) Int. Cl. C12Q 1/6886; G16H 10/40; G16H 20/10

(52) U.S. Cl. CPC C12Q 1/6886; G16H 10/40; G16H 20/10; C12Q 2600/106; C12Q 2600/158

(57) ABSTRACT

The present disclosure relates to methods and systems for characterizing cancer testis antigen burden (“CTAB”), for predicting cancer survival outcomes using CTAB analysis, and for recommending and/or treating cancer using CTAB analysis. Particularly, aspects are directed to measuring expression of a panel of cancer testis antigen (CTA) gene markers in a tissue form a tumor, determining a CTAB based on the measured expression of the CTA gene markers, predicting response of the tumor to immune checkpoint blockade therapy based on the determined CTAB, where the determined CTAB is associated with a predicted favorable response of the tumor to immune checkpoint blockade therapy when the CTAB is ≥171, determining an immune checkpoint blockade therapy for the tumor based on the predicted response of the tumor to immune checkpoint blockade therapy, and administering the determined immune checkpoint blockade therapy to the patient.

### CROSS-REFERENCE TO RELATED APPLICATIONS

The present application claims priority and benefit from U.S. Provisional Application No. 63/263,913, filed on Nov. 11, 2021, the entire contents of which are incorporated herein by reference for all purposes.

### FIELD

The present disclosure is directed generally to methods and systems for characterizing cancer testis antigen burden (“CTAB”), for predicting cancer survival outcomes using CTAB analysis, and for recommending and/or treating cancer using CTAB analysis.

### BACKGROUND

Cancer testis antigens (CTAs) have highly tissue-restricted expression under normal gene regulation, being expressed almost exclusively in male germ cells. When the normal gene regulation is disrupted, such as in malignancies, CTAs may become expressed in a diverse variety of somatic tissues. When expressed in cancer cells, CTAs are highly immunogenic and have the capacity to elicit cancer-specific immune responses in diverse malignancies. Consequently, CTAs have become a prime target of natural T cell response, immune cell-based therapies, immune checkpoint inhibitors (ICIs) or immune checkpoint blockades (ICBs), and cancer vaccines.

ICIs have emerged as effective treatments in a variety of cancers, including breast cancers, colorectal cancers, and lung cancers like non-small cell lung cancer (NSCLC). NSCLC accounts for ˜50% of brain metastases. While the clinical utility of single agent ICI or in combination with chemotherapy has been well established, ICI-based immunotherapy has been estimated to cost USD 100,00-250,000 per patient. Consequently, there remains an unmet need for the development of biomarkers that can better predict patients' response to ICI treatment.

### SUMMARY

The present disclosure is directed to a method of for characterizing patient response to ICI treatment. Co-expression of multiple CTA genes occurs in many tumor types and can be reliably detected using a targeted RNA-seq approach. Utilization of this co-expression pattern to calculate Cancer Testis Antigen Burden (CTAB) reveals tumor-type associated signatures, which in a small NSCLC cohort are associated with the overall survival (OS). These immunogenic antigens expose the tumor cells to natural or immunotherapy augmented cell-based immune response, and thus CTAB is a predictive marker for therapeutic response to ICIs.

In various embodiments, a method is provided for characterizing response of a patient's tumor to immune checkpoint blockade therapy. The method comprises: (a) obtaining tissue from a tumor; (b) measuring expression of a panel of cancer testis antigen (CTA) gene markers in the tissue; (c) determining a cancer testis antigen burden (CTAB) based on the measured expression of the CTA gene markers; (d) predicting response of the tumor to immune checkpoint blockade therapy based on the determined CTAB, where the determined CTAB is associated with a predicted favorable response of the tumor to immune checkpoint blockade therapy when the CTAB is ≥171; (e) determining an immune checkpoint blockade therapy for the tumor based on the predicted response of the tumor to immune checkpoint blockade therapy; and (f) administering the determined immune checkpoint blockade therapy to the patient.

In various embodiments, a diagnostic test is provided for characterizing, using a panel of CTA genes, a CTAB of a tumor. The diagnostic test comprises the steps of: (a) obtaining tissue from the tumor; (b) measuring expression of a panel of cancer testis antigen (CTA) gene markers in the tissue; (c) determining the CTAB based on the measured expression of CTA gene markers; (d) characterizing the tumor as high-CTAB when CTAB is ≥171 and low-CTAB when CTAB <170; and (e) predicting a favorable response of the tumor to immune checkpoint blockade therapy when the tumor is high-CTAB and a less-favorable response of the tumor to immune checkpoint blockade therapy when the tumor is low-CTAB.

In various embodiments, a method is provided for that comprises: (a) measuring expression of a panel of cancer testis antigen (CTA) gene markers in a tissue form a tumor; (b) determining a cancer testis antigen burden (CTAB) based on the measured expression of the CTA gene markers; (c) predicting response of the tumor to immune checkpoint blockade therapy based on the determined CTAB, where the determined CTAB is associated with a predicted favorable response of the tumor to immune checkpoint blockade therapy when the CTAB is ≥171; and (d) determining an immune checkpoint blockade therapy for the tumor based on the predicted response of the tumor to immune checkpoint blockade therapy

In some embodiments, the expression of the CTA gene markers is measured by RNA-seq.

In some embodiments, the tumor is non-small cell lung cancer (NSCLC).

In some embodiments, the panel of CTA gene markers comprises XAGE1B, SSX2, MLANA, MAGEC2, MAGEA12, MAGEA10, MAGEA4, MAGEA3, MAGEA1, GAGE13, GAGE12J, GAGE10, GAGE2C, CTAG2, CTAG1B, and BAGE.

In some embodiments, the immune checkpoint blockade therapy comprises one or more of nivolumab, pembrolizumab, ipilimumab, atezolizumab, and durvalumab.

In some embodiments, a system is provided that includes one or more data processors and a non-transitory computer readable medium containing instructions which, when executed on the one or more data processors, cause the one or more data processors to perform part or all of one or more methods or processes disclosed herein.

In some embodiments, a computer-program product is provided that is tangibly embodied in a non-transitory machine-readable medium and that includes instructions configured to cause one or more data processors to perform part or all of one or more methods disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The present invention will be better understood in view of the following non-limiting figures, in which:

FIG. 1 shows a representation of the 5,450 samples from clinically tested FFPE tumors spanning 39 cancer types in accordance with various embodiments.

FIG. 2 shows a flowchart showing calculation of gene expression normalized reads per million (nRPM) from raw absolute read count values in the discovery cohort in accordance with various embodiments.

FIG. 3 shows a gene expression across all tumors for 17 CTAs classified as Positive (nRPM≥20) or Negative (nRPM<20) in accordance with various embodiments.

FIG. 4 shows CTAB distributions in A) discovery, B) TCGA, and C) retrospective cohorts in accordance with various embodiments.

FIG. 5 shows cancer type CTAB distributions in A) discovery, B) TCGA, and C) retrospective cohorts in accordance with various embodiments

FIG. 6 shows CoxPH regression analysis for CTAB, age, and gender effects in A) TCGA, and B) retrospective cohorts in accordance with various embodiments.

FIG. 7 shows Kaplan-Meier survival analysis comparing CTAB positive (≥171) and negative (<171) groups for A) TCGA and B) retrospective cohorts in accordance with various embodiments.

FIG. 8 shows co-expression pattern of 15 cancer testis antigens across the entire 5450 tumors of the DC in accordance with various embodiments. Pairwise Pearson correlation values are indicated in each grid square and colored according to the correlation value, as indicated by the shaded bar at right. A black “X” through a square denotes a non-significant (p>0.05) correlation, and the black squares about the chart diagonal denote clusters of co-expressing CTA.

FIG. 9 shows violin plots detailing the distributions of the cancer testis antigen burden (CTAB) in each of the 39 histologies represented in the 5450-sample DC in accordance with various embodiments.

FIG. 10 shows PCA and hierarchical clustering results: A) eigenvector plot detailing the influence of five constituent biomarkers on the first two principle components; B) classification of the 110 patient NSCLC validation cohort into four phenotypes; and C) distribution of the four phenotypes within the validation cohort in accordance with various embodiments.

FIG. 11 shows Kaplan-Meier survival analyses for NSCLC validation cohort (n=110) as stratified by: A) PD-L1 IHC status; B) TMB status; C) cell proliferation; D) tumor immunogenic signature (TIGS); E) CTAB; and F) phenotype in accordance with various embodiments.

FIG. 12 shows CoxPH survival analysis of phenotype, the five constituent biomarkers, and sex and race as survival predictors in accordance with various embodiments.

FIG. 13 shows disease control rate (DCR) for the 110 NSCLC patient validation cohort as subdivided into the four phenotypes: tumor dominant; proliferative; inflamed; and checkpoint in accordance with various embodiments.

In the appended figures, similar components and/or features can have the same reference label. Further, various components of the same type can be distinguished by following the reference label by a dash and a second label that distinguishes among the similar components. If only the first reference label is used in the specification, the description is applicable to any one of the similar components having the same first reference label irrespective of the second reference label.

### DETAILED DESCRIPTION

The ensuing description provides preferred exemplary embodiments only, and is not intended to limit the scope, applicability or configuration of the disclosure. Rather, the ensuing description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

Also, it is noted that individual embodiments may be described as a process which is depicted as a flowchart, a flow diagram, a data flow diagram, a structure diagram, or a block diagram. Although a flowchart or diagram may describe the operations as a sequential process, many of the operations may be performed in parallel or concurrently. In addition, the order of the operations may be re-arranged. A process is terminated when its operations are completed, but could have additional steps not included in a figure. A process may correspond to a method, a function, a procedure, a subroutine, a subprogram, etc. When a process corresponds to a function, its termination may correspond to a return of the function to the calling function or the main function.

### Techniques for Characterizing CTAB

This invention is directed to methods and systems for predicting ICI response and survival in patients with tumors across multiple histologies, and particularly in NSCLC.

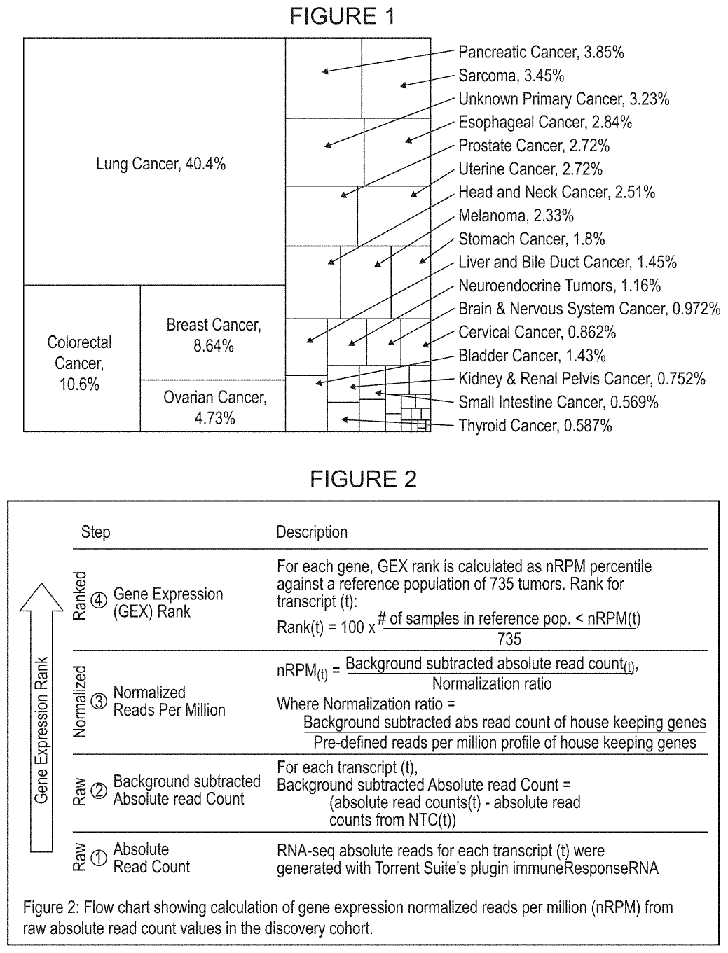

In one embodiment, a pan-cancer discovery cohort (DC) was studied to develop a low- and high-CTAB cutoff. Standard-of-care comprehensive genomic and immune profiling was performed on 5450 FFPE tumors representing 39 histologic types, assessing expression levels of 395 immune genes and >500 tumor-associated genes. Inclusion criteria for the samples was based on clinical QC parameters for RNA-seq. As shown in FIG. 1, the DC primarily consists of lung cancer (40.4%) followed by colorectal (10.6%), breast (8.64%), and ovarian cancer (4.73%), with other cancer types in lesser percentages. Targeted RNA-seq was performed on the 5450 FFPE tumors. From the gene expression data of the DC, three gene expression signatures were calculated: cell proliferation (CP), tumor immunogenic signature (TIGS), and cancer testis antigen burden (CTAB). PD-L1 status of each tumor was assessed by IHC, and tumor mutational burden (TMB) was calculated.

In a second embodiment, a cohort from The Cancer Genome Atlas (TCGA) was used to validate the classifier based on CTAB distribution and serve as a non-ICB-treated population. The TCGA included 19923 tumors across 32 tumor types. Subsequently, in a third embodiment, a nearest neighbor method was used to classify an ICI treated 242-patient retrospective validation cohort (RC) including 110 NSCLC patients as well as patients with melanoma and renal cell carcinoma.

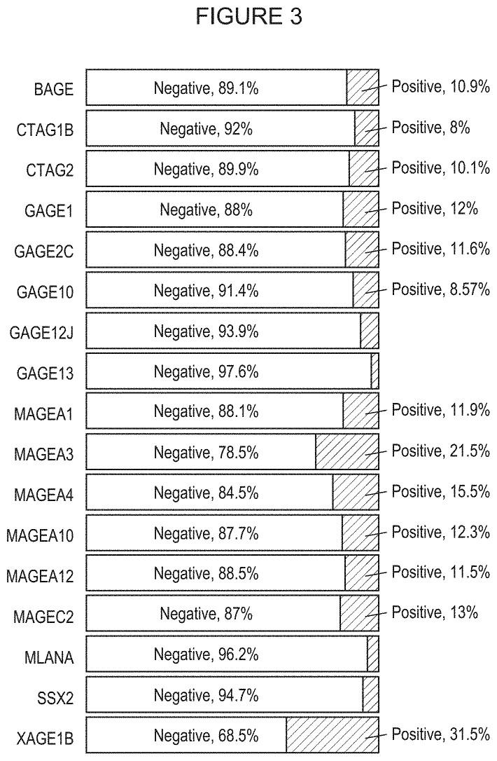

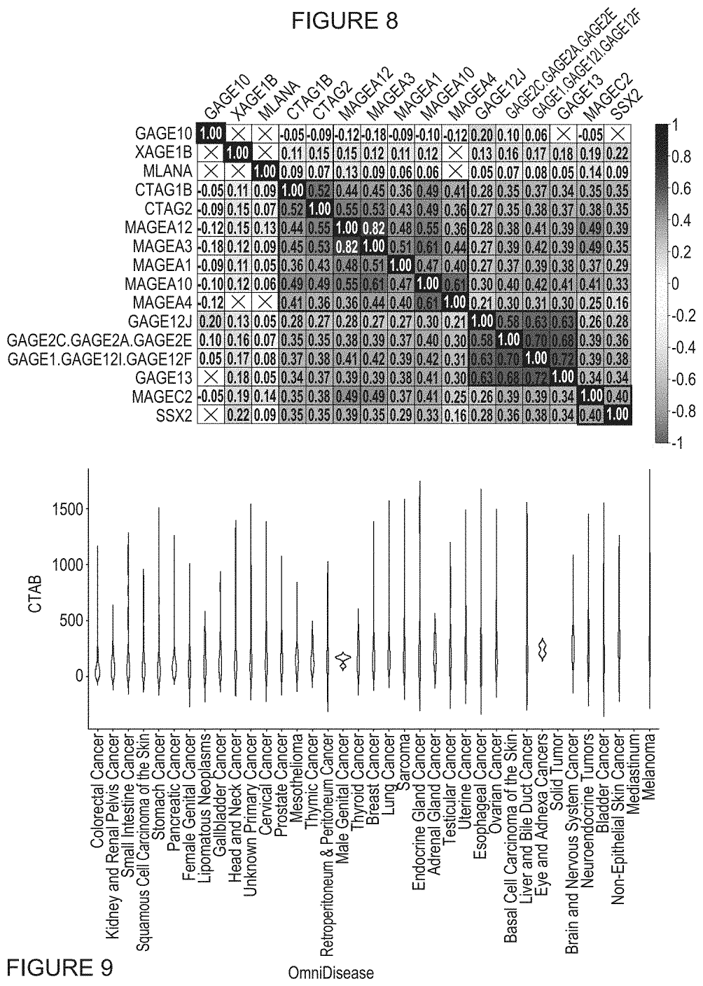

Using an amplicon-based NGS approach, the expression levels of 17 CTAs were ranked against a pan-cancer reference population. The method for calculating gene expression normalized reads from raw absolute read count and calculating the Gene Expression (GEX) rank is shown in FIG. 2. As shown in FIG. 3, the CTAs measured are XAGE1B, SSX2, MLANA, MAGEC2, MAGEA12, MAGEA10, MAGEA4, MAGEA3, MAGEA1, GAGE13, GAGE12J, GAGE10, GAGE2C, CTAG2, CTAG1B, and BAGE. Positive CTA expression prevalence ranged from 3% (GAGE13) to 31.5% (XAGE1B) across the 5450 tumors studied. The Co-expression pattern of 15 of the CTAs across the entire 5450 tumors in the DC is shown in FIG. 8. Co-expression of CTAs was observed across several histologies, with the highest levels of co-expression observed within the MAGEA and GAGE families.

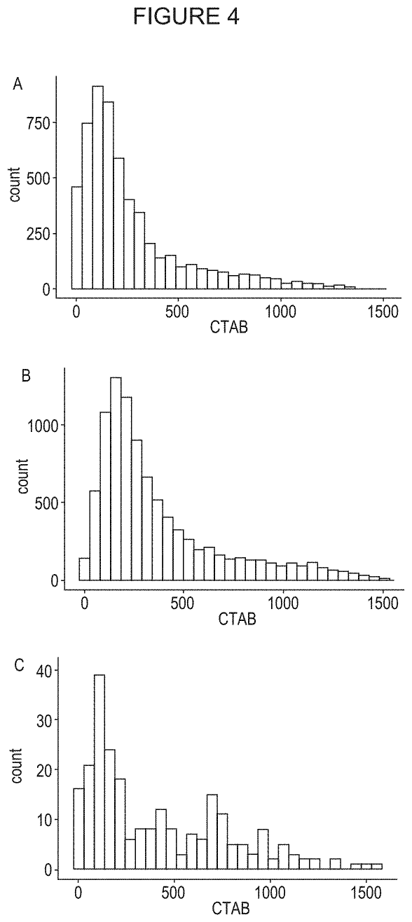

CTAB was calculated for each sample, cohort and tumor type as the sum of the 17 CTA gene expression ranks. The results of each cohort CTAB composition is shown below in Table 1. As shown in FIG. 4, the three cohorts each demonstrated overlapping single-peak, left-skewed CTAB distribution curves centered at CTAB values between 171 for the DC ad 256 for RC. The DC median CTAB of 171 was used to classify all three cohorts into high- and low-CTAB groups.

TABLE 1
Cohort CTAB composition

| | | Median | N Positive | N Negative |
|---|---|---|---|---|
| Cohort | N | CTAB | (CTAB ≥ 171) | (CTAB < 171) |
| DC | 5450 | 171 | 2806 | 2828 |
| TCGA | 19923 | 254 | 6413 | 2860 |
| RC | 242 | 256 | 148 | 94 |

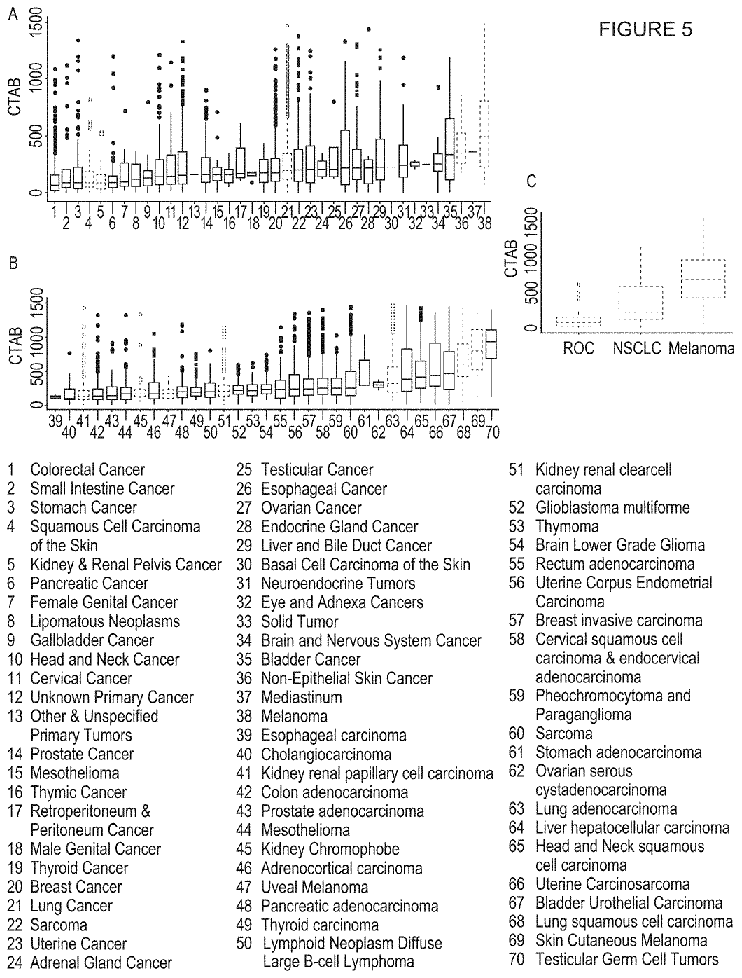

As shown in FIGS. 5 and 9, when grouped by tumor types and ordered by median CTAB, the CTAB distributions for tumor types within the three cohorts were comparable. CTAB values ranged from 0-1700, with kidney cancer demonstrating overall lowest mean CTAB (110) and melanoma the highest (550). NSCLC had an average CTAB of 283.

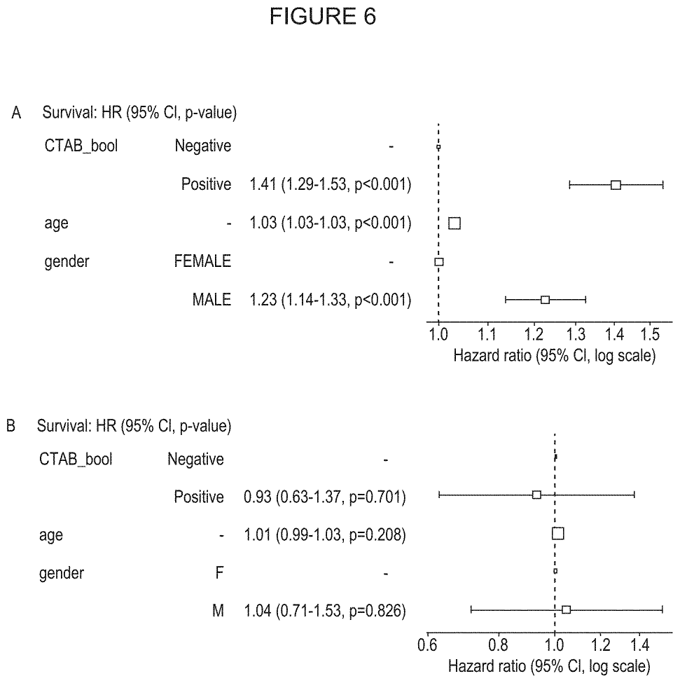

As shown in FIG. 6, OS analysis was performed on the TCGA and ICB-treated cohorts using a CoxPH regression model to determine the Hazard Ratio (HR). The CoxPH regression analysis revealed an association between the CTAB threshold classifier and OS in both the ICB-treated RC and non-ICB-treated TCGA. However, the direction of this association differed between the two cohorts, with high-CTAB samples having better survival (HR=0.936, p=0.076) in the ICB-treated RC and worse survival (HR: 1.007, p=0.084) in the non-ICB-treated TCGA.

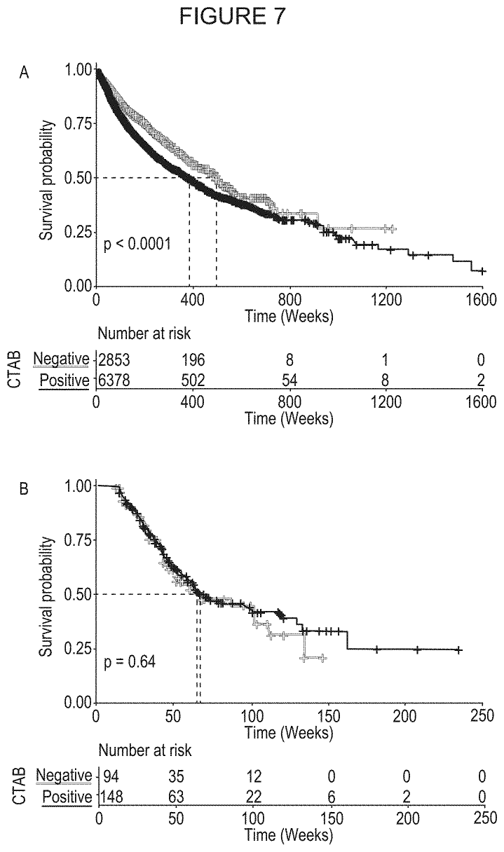

Kaplan-Meier survival analyses comparing CTAB positive (≥171) and negative (<171) groups for TCGA and RC Kaplan-Meier revealed a strong association (p<0.000) between positive CTAB status and worse survival in the TCGA, as show in FIG. 7. This association did not exist in the RC (p=0.64), though positive CTAB status trended toward better survival. This difference suggests that advances in immunotherapy targeting CTA have largely eliminated the survival disbenefit observed in the pre-immunotherapy TCGA.

The CTAB distribution is maintained across the DC and TCGA, as well as across a wide range of tumor types, supporting CTAB as valid and histology agnostic classifier. Additionally, when evaluating the ICB-treated and non-ICB-treated cohorts, CTAB demonstrated the ability to predict OS, pointing to the utility of ICB in supporting CTA-specific natural immune response.

The DC was further evaluated by comprehensive genomic and immune profiling of the tumor immune microenvironment. Individual and combination biomarker assessment included PD-L1 IHC, TMB, tumor inflammation (TIGS), cell proliferation (CP) and cancer testis antigen burden (CTAB). From the DC, combinations of molecular and immune biomarkers were identified and applied to a sub-cohort of the RC, 110 metastatic NSCLC patients treated with pembrolizumab+chemo or pembrolizumab alone to correlate with response. Comparison of objective response rates (ORR) was performed using Chi-square test. Kaplan-Meir analysis was performed to test for differences in OS and 1-year OS.

Example 1. In the 110-samples from NSCLC patients who were ICB-treated from 242-sample sub-cohort, High CTAB showed better OS compared to Low CTAB (HR: 0.55, p=0.07). Additionally, when combined with tumor inflammation and cell proliferation biomarkers, highly inflamed but poorly proliferative tumors with High CTAB had improved OS (HR: 0.27, p=0.05). A significant association with higher response (HR=1.84; p=0.05) was detected for the entire 242-sample RC.

TABLE 2
Overall Survival (OS) and objective response rate (ORR) differences
for the entire retrospective cohort for High versus Low CTAB.

Overall Survival

| | | | Hazard | | | |
|---|---|---|---|---|---|---|
| Group | Predictor | Survival Comparison | Ratio | CI Low | CI High | P Value |
| Retrospective Cohort | CTAB MEDIAN | Above Median vs Below Median | 0.63 | 0.43 | 0.94 | 0.02 |
| (n = 242) | | | | | | |
| NSCLC (n = 110) | CTAB MEDIAN | Above Median vs Below Median | 0.5 | 0.29 | 1.04 | 0.07 |
| NSCLC (N = 110) | CTAB MEDIAN | TIGS = Strong CP = High | 0.28 | 0.03 | 2.69 | 0.27 |
| | | TIGS = Strong CP = Moderate | 0.66 | 0.20 | 2.20 | 0.49 |
| | | TIGS = Strong CP = Poor | 0.27 | 0.08 | 0.99 | 0.05 |

| | | | Hazard | | | |
|---|---|---|---|---|---|---|
| Group | Predictor | ORR Comparison | Ratio | CI Low | CI High | P Value |
| Retrospective Cohort | CTAB MEDIAN | Above Median vs Below Median | 1.84 | 1.07 | 3.36 | 0.05 |
| (n = 242) | | | | | | |
| NSCLC (N = 110) | CTAB MEDIAN | TIGS = Moderate CP = High | 0.66 | 0.03 | 14.05 | 0.79 |
| | | TIGS = Moderate CP = Moderate | 0.00 | 0.00 | NA | 0.98 |
| | | TIGS = Moderate CP = Poor | 6.86 | 0.83 | 56.60 | 0.07 |

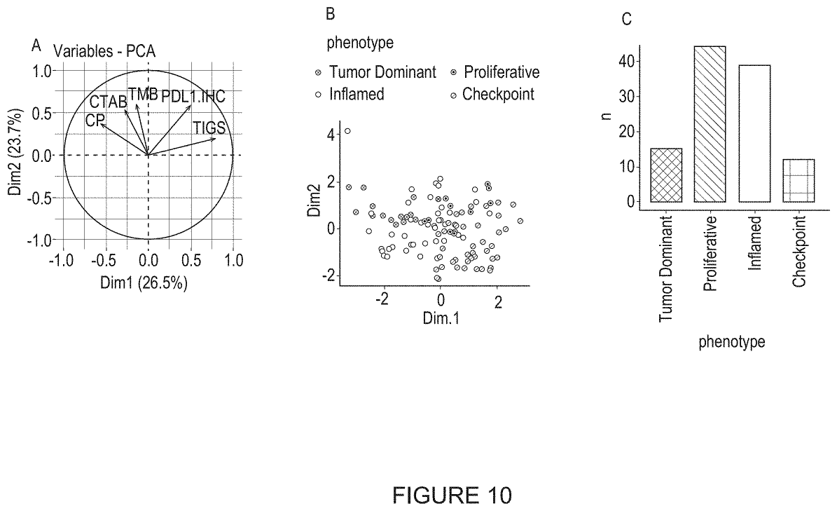

As showing in FIG. 10, analysis of the DC revealed four distinct biomarker combination groups that describe underlying tumor immunobiology: tumor dominant (CTAB, TMB, CP High), proliferative (CP High), inflamed (TIGS High), and checkpoint (PDL1, TIGS and TMB High). Application of these biomarker groups to the RC demonstrated significant differences in response to ICI regimens between groups (p=0.04). Patients in the proliferative group (35.1%, 79/225; median PD-L1=20% TPS) treated with single agent pembrolizumab showed a significantly higher ORR (59%; 16/27) compared to pembrolizumab+chemo (27%; 14/52; p=0.005), significantly improved 1-yr OS (p=0.03), and trend towards better OS (p=0.14). Importantly, patients in the inflamed group (16%, 36/225; median PD-L1=1% TPS), suggested that pembrolizumab+chemo (ORR 26.1%; 6/23) was not associated with ORR compared to pembrolizumab (ORR 31%; 4/13, p=0.76), or OS (p=0.37) and 1-yr OS (p=0.57). Consequently PD-L1 low NSCLC patients with a proliferative phenotype may benefit from single agent pembrolizumab, whereas PD-L1 low cases with an inflamed phenotype may benefit from both single agent and combination pembrolizumab.

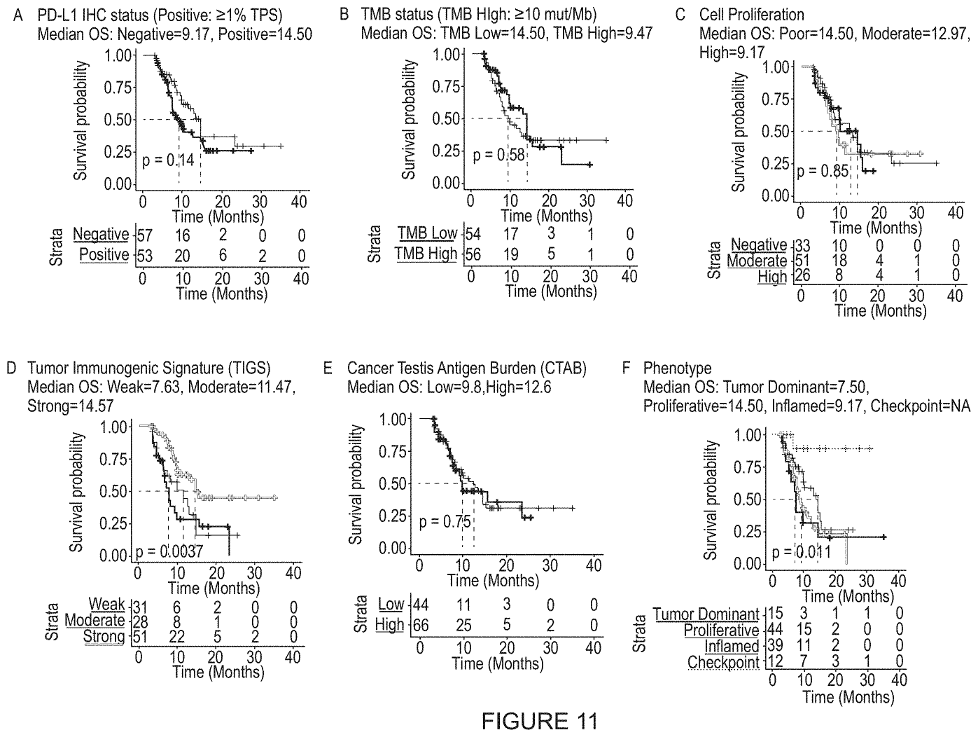

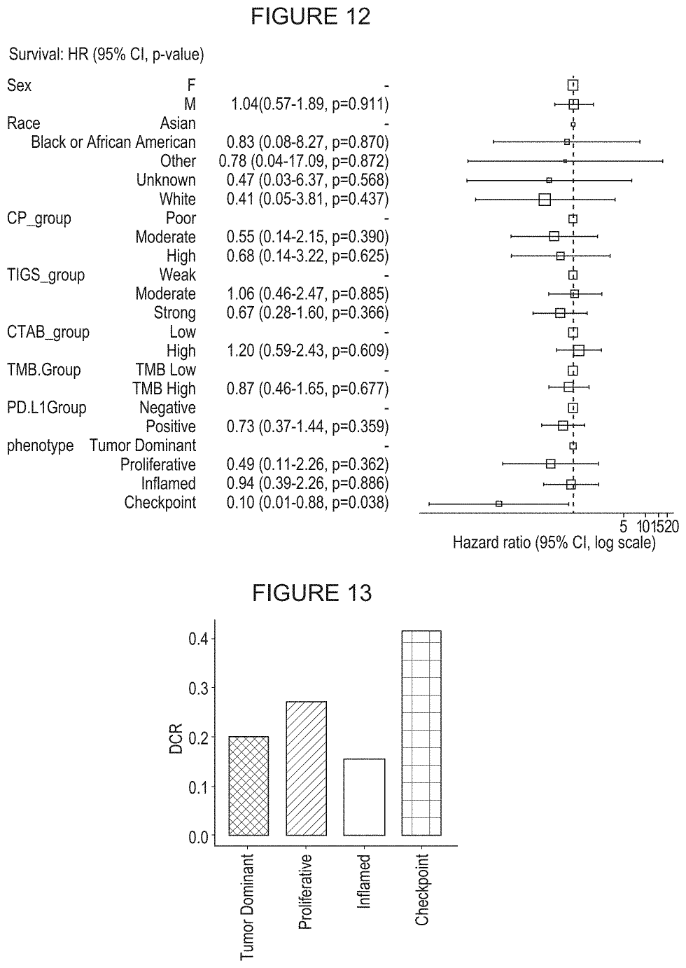

The association between these groups and ICI treatment response was determined by overrepresentation analysis, and overall survival was assessed using Kaplan-Meyer and CoxPH analyses, as shown in FIGS. 11 and 12. Kaplan-Meier survival analysis suggested a significant relationship between these groups and overall survival [p=0.035], with the proliferative and checkpoint groups demonstrating increased survival over tumor-dominant and inflamed groups. Phenotype stratification demonstrated greater increase in median survival than stratification by any constituent biomarker. CoxPH analysis showed the checkpoint group to have a significantly decreased hazard ratio [HR=0.10; p=0.038] for ICI treatment. In all Kaplan-Meier and CoxPH analyses, this checkpoint group outperformed any of its constituent biomarkers as a survival predictor.

Furthermore, as shown in FIG. 13, significant association with ICI response was found by classifying the RC into the four groups, with the following results: tumor dominant [DCR=0.200]; proliferative [DCR=0.273], inflamed [DCR=0.154]; and checkpoint [DCR=0.417]. The checkpoint group was overrepresented by the highest proportion of disease control [p=0.0313].

An integrated approach combining comprehensive tumor profiling and emerging biomarkers better predicts ICI response and survival in multiple histologies. Divergent outcomes between the resulting groups are likely the result of distinct tumor-immune interaction modalities.

### Additional Considerations

Specific details are given in the above description to provide a thorough understanding of the embodiments. However, it is understood that the embodiments can be practiced without these specific details. For example, circuits can be shown in block diagrams in order not to obscure the embodiments in unnecessary detail. In other instances, well-known circuits, processes, algorithms, structures, and techniques can be shown without unnecessary detail in order to avoid obscuring the embodiments.

Implementation of the techniques, blocks, steps and means described above can be done in various ways. For example, these techniques, blocks, steps and means can be implemented in hardware, software, or a combination thereof. For a hardware implementation, the processing units can be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above, and/or a combination thereof.

Also, it is noted that the embodiments can be described as a process which is depicted as a flowchart, a flow diagram, a data flow diagram, a structure diagram, or a block diagram. Although a flowchart can describe the operations as a sequential process, many of the operations can be performed in parallel or concurrently. In addition, the order of the operations can be re-arranged. A process is terminated when its operations are completed, but could have additional steps not included in the figure. A process can correspond to a method, a function, a procedure, a subroutine, a subprogram, etc. When a process corresponds to a function, its termination corresponds to a return of the function to the calling function or the main function.

Furthermore, embodiments can be implemented by hardware, software, scripting languages, firmware, middleware, microcode, hardware description languages, and/or any combination thereof. When implemented in software, firmware, middleware, scripting language, and/or microcode, the program code or code segments to perform the necessary tasks can be stored in a machine-readable medium such as a storage medium. A code segment or machine-executable instruction can represent a procedure, a function, a subprogram, a program, a routine, a subroutine, a module, a software package, a script, a class, or any combination of instructions, data structures, and/or program statements. A code segment can be coupled to another code segment or a hardware circuit by passing and/or receiving information, data, arguments, parameters, and/or memory contents. Information, arguments, parameters, data, etc. can be passed, forwarded, or transmitted via any suitable means including memory sharing, message passing, ticket passing, network transmission, etc.

For a firmware and/or software implementation, the methodologies can be implemented with modules (e.g., procedures, functions, and so on) that perform the functions described herein. Any machine-readable medium tangibly embodying instructions can be used in implementing the methodologies described herein. For example, software codes can be stored in a memory. Memory can be implemented within the processor or external to the processor. As used herein the term “memory” refers to any type of long term, short term, volatile, nonvolatile, or other storage medium and is not to be limited to any particular type of memory or number of memories, or type of media upon which memory is stored.

Moreover, as disclosed herein, the term “storage medium”, “storage” or “memory” can represent one or more memories for storing data, including read only memory (ROM), random access memory (RAM), magnetic RAM, core memory, magnetic disk storage mediums, optical storage mediums, flash memory devices and/or other machine-readable mediums for storing information. The term “machine-readable medium” includes, but is not limited to portable or fixed storage devices, optical storage devices, wireless channels, and/or various other storage mediums capable of storing that contain or carry instruction(s) and/or data.

While the principles of the disclosure have been described above in connection with specific apparatuses and methods, it is to be clearly understood that this description is made only by way of example and not as limitation on the scope of the disclosure.

What is claimed is:

1. A method for characterizing response of a patient's tumor to immune checkpoint blockade therapy, the method comprising the steps of:
(a) obtaining tissue from the tumor;
(b) measuring expression of a panel of cancer testis antigen (CTA) gene markers in the tissue;
(c) determining a cancer testis antigen burden (CTAB) based on the measured expression of the CTA gene markers;
(d) predicting response of the tumor to immune checkpoint blockade therapy based on the determined CTAB,
wherein the determined CTAB is associated with a predicted favorable response of the tumor to immune checkpoint blockade therapy when the CTAB is ≥171;
(e) determining an immune checkpoint blockade therapy for the tumor based on the predicted response of the tumor to immune checkpoint blockade therapy; and
(f) administering the determined immune checkpoint blockade therapy to the patient.

2. A diagnostic test for characterizing, using a panel of cancer testis antigen (CTA) genes, a cancer testis antigen burden (CTAB) of a tumor, the diagnostic test comprising the steps of:
(a) obtaining tissue from the tumor;
(b) measuring expression of the panel of CTA gene markers in the tissue;
(c) determining the CTAB based on the measured expression of CTA gene markers;
(d) characterizing the tumor as high-CTAB when CTAB is ≥171 and low-CTAB when CTAB <170; and
(e) predicting a favorable response of the tumor to immune checkpoint blockade therapy when the tumor is high-CTAB and a less-favorable response of the tumor to immune checkpoint blockade therapy when the tumor is low-CTAB.

3. The method of claim 1, wherein the expression of the CTA gene markers is measured by RNA-seq.

4. The method of claim 1, wherein the tumor is non-small cell lung cancer (NSCLC).

5. The method of claim 1, wherein the panel of CTA gene markers comprises XAGE1B, SSX2, MLANA, MAGEC2, MAGEA12, MAGEA10, MAGEA4, MAGEA3, MAGEA1, GAGE13, GAGE12J, GAGE10, GAGE2C, CTAG2, CTAG1B, and BAGE.

6. The method of claim 1, wherein the immune checkpoint blockade therapy comprises one or more of nivolumab, pembrolizumab, ipilimumab, atezolizumab, and durvalumab.

7. A system comprising:
one or more data processors; and
a non-transitory computer readable medium storing instructions which, when executed on the one or more data processors, cause the one or more data processors to perform:
(a) measuring expression of a panel of cancer testis antigen (CTA) gene markers in a tissue form a tumor;
(b) determining a cancer testis antigen burden (CTAB) based on the measured expression of the CTA gene markers;
(c) predicting response of the tumor to immune checkpoint blockade therapy based on the determined CTAB, wherein the determined CTAB is associated with a predicted favorable response of the tumor to immune checkpoint blockade therapy when the CTAB is ≥171; and
(d) determining an immune checkpoint blockade therapy for the tumor based on the predicted response of the tumor to immune checkpoint blockade therapy.

8. The system of claim 7, wherein the expression of the CTA gene markers is measured by RNA-seq.

9. The system of claim 7, wherein the tumor is non-small cell lung cancer (NSCLC).

10. The system of claim 7, wherein the panel of CTA gene markers comprises XAGE1B, SSX2, MLANA, MAGEC2, MAGEA12, MAGEA10, MAGEA4, MAGEA3, MAGEA1, GAGE13, GAGE12J, GAGE10, GAGE2C, CTAG2, CTAG1B, and BAGE.

11. The system of claim 7, wherein the immune checkpoint blockade therapy comprises one or more of nivolumab, pembrolizumab, ipilimumab, atezolizumab, and durvalumab.
